(19) United States
(12) Patent Application Publication
(10) Pub. No.: US 2017/0296378 A1
(43) Pub. Date: Oct. 19, 2017

(54) Therapeutic Room Thermostat

(71) Applicant: Joseph H. Schaefer, Grafton, WI (US)

(72) Inventor: Joseph H. Schaefer, Grafton, WI (US)

(21) Appl. No.: 15/099,888

(22) Filed: Apr. 15, 2016

(51) Int. Cl. A61F 7/00; G05B 15/02; F24F 11/00

(52) U.S. Cl. CPC A61F 7/0053; G05B 15/02; F24F 11/006; A61F 2007/0096; A61F 2007/0086

(57) ABSTRACT

The purpose of the disclosed thermostat is to prevent age-related diseases, induced by long term cool indoor temperatures. As people age, the body produces progressively less heat while aging-impaired vasoconstriction results in progressively more heat being lost. This age-induced cold stress requires ever greater use of vasoconstriction or behavior to maintain the body's heat balance. When behavioral regulation becomes diminished with age, vasoconstriction becomes progressive throughout life. In at risk elderly, an ongoing mild indoor cold stress can unknowingly maximize negative feedback vasoconstriction leaving it unable to further defend core body temperature. Before hypothermia occurs, positive feedback vasoconstriction activates as a defense mechanism. Like inflammation and fever, this beneficial defense mechanism can also cause harm when its use becomes excessive. The therapeutic function of this medical device is to sense skin temperature and use it to modify the indoor environment, keeping thermoregulation within the effective range of negative feedback vasoconstriction.

### CROSS-REFERENCE TO RELATED APPLICATIONS

Not applicable.

### STATEMENT CONCERNING FEDERALLY SPONSORED RESEARCH OR DEVELOPMENT

Not applicable.

### FIELD OF INVENTION

This disclosure relates to a thermoregulation-assistive system involving a thermostat and a temperature sensor for placement on a user in which the sensor provides temperature information to the thermostat for therapeutic purposes.

### BACKGROUND

Heat balance is maintained by the body through both involuntary physiological thermoregulation and behavioral thermoregulation. Behavioral regulation enables tropical humans to live in extreme climates, but does not provide fine control of heat balance. Physiological regulation provides this fine control but is only effective within a relatively narrow range of ambient temperatures. At set-point, the heat production of the body is in balance with its heat loss to the environment. This heat balance is mainly regulated by the skin and the heat lost depends on a gradient between skin temperature and environmental temperature. Each physiological thermoregulatory response has its own activation threshold temperature and maximum intensity. There is an orderly progression of responses, and response intensities are in proportion to need.

Voluntary behavioral thermoregulation is driven by the human body's conscious perception of comfort and discomfort. Some examples of voluntary behavioral regulation to cold include, but are not limited to, dressing more warmly, raising the thermostat, moving to a warmer location, and voluntary movement.

Behavioral regulation can vary between individuals, for different reasons. The senses diminish with age leaving the aged with a diminished ability to perceive the cold. This diminished ability to perceive and respond to cold may be further exacerbated by dementia or Alzheimer's disease in some individuals. Diabetics with peripheral neuropathy can lose the cold sensations necessary for adequate behavioral regulation. Some elderly have impaired or habituated shivering mechanisms. Many individuals are concerned with saving on their heating costs, unaware of any possible harm. Some individuals sleep in cold bedrooms. Others live with cooler temperature settings preferred by their spouses or co-workers.

Accordingly, behavioral regulation is best suited for wide variations of temperature, typically needed outdoors, while it is least suited for providing fine control of heat balance, typically needed indoors. In contrast, in a mildly cool indoor environment, heat balance is best maintained through the use of vasoconstriction.

Cutaneous vasoconstriction is the initial thermoregulatory response to skin cooling, effectively minimizing heat loss to the environment. Vasoconstriction reduces heat loss and defends core body temperature, but at the expense of a decline in skin temperature. Vasoconstriction is most often generalized, which is inaccurate. Vasoconstriction exists in two distinct and differently configured forms. Whole-body skin cooling stimulates a homeostatic response named reflex-mediated vasoconstriction (reflex vasoconstriction). This systemic response decreases the skin temperature of the entire periphery. Local skin cooling stimulates a non-reflex response named locally-mediated vasoconstriction (local vasoconstriction). This local response decreases the skin temperature at the local site where it occurs. Each of the two responses reduces heat loss and defends core body temperatures, at the expense of a decline in skin temperature. These two mechanisms are not mutually exclusive and often interact together during cold exposure to maximize vasoconstriction. When both responses take place simultaneously at a local site, the declines in skin temperature become cumulative, at the expense of a colder local skin temperature and a significant reduction in local blood flow.

When vasoconstriction is generalized, it usually defines the better known and understood reflex vasoconstriction. Reflex vasoconstriction is controlled with negative feedback and, as such, is a dynamic response. Negative feedback mechanisms oppose the sensed change to regulate or maintain physiological functions within a set and narrow range, that is, a drop in sensed core body temperature increases the intensity of reflex vasoconstriction, which then raises core body temperature. Homeostasis for many of the control mechanisms of the body is maintained by using negative feedback. Reflex vasoconstriction is also a graded response where the intensity of the response mirrors the intensity of the whole-body cold stimulus until blood flow reaches a basement plateau, after which further cooling will not induce further constriction. When reflex vasoconstriction becomes maximal, the mean skin temperature is about 31 degrees Centigrade. As a dynamic response, reflex vasoconstriction is in a state of constant change, striving to regulate or maintain the homeostatic heat balance of the body, until it reaches its maximum response intensity and can no longer remain effective.

### SUMMARY

According to one aspect of the invention, a medical device is disclosed to maintain thermoregulation within an effective range of reflex-mediated vasoconstriction for an individual when the individual is subjected to a mild indoor cold stress. The medical device includes a remote toe temperature sensor and a dual input thermostat. The remote toe temperature sensor is for placement on or near a toe of the individual and provides a toe temperature. The dual input thermostat is adapted for connection to an HVAC (Heating, venting, and/or air conditioning) system for adjusting an air temperature in an operational zone to a set point temperature and is further adapted for connection to the remote toe temperature sensor to receive the toe temperature. Moreover, dual input thermostat includes at least one control for the adjustment of a high limit room temperature setting and a low limit room temperature setting to delimit a range of temperature operation for the HVAC system and further includes an air temperature sensor providing the air temperature within the operational zone. The dual input thermostat is configured to receive temperature inputs from at least the remote toe temperature sensor and the air temperature sensor and is configured to adjust the set point temperature of the HVAC system relative to the air temperature inversely and proportionately based on the toe temperature provided by the remote toe temperature sensor. The set point temperature is bounded by the range of temperature operation for the HVAC system delimited by the high limit room temperature setting and low limit room temperature setting of the dual input thermostat (and is therefore only adjustable within that range).

In some forms, the remote toe temperature sensor in communication with the dual input thermostat may be in wireless communication with the dual input thermostat.

In some forms, the control(s) for the adjustment of the high limit room temperature setting and the low limit room temperature may include a pair of controls including one control for controlling the high limit room temperature setting and one control for controlling the low limit room temperature setting.

These controls may be disposed on a housing of the dual input thermostat.

In some forms, the air temperature sensor may be located within the dual input thermostat.

In some forms, the dual input thermostat may be configured such that, when the dual input thermostat receives a temperature input from the toe temperature sensor, an adjustment the set point temperature of the HVAC system relative to the air temperature is time delayed to dampen the response to the toe temperature sensor. The dual input thermostat may further include a visual indicator (for example, a colored light such as a light emitting diode) configured to indicate a warning when dampened toe temperature falls below a predetermined value corresponding to the skin activation temperature of locally-mediated vasoconstriction.

In some forms, the medical device may protect the individual, regardless of age of the individual, against one or more of indoor cold-induced disease, indoor cold-induced hypertension, an age-related progression of vasoconstriction, indoor positive feedback vasoconstriction, and indoor hypothermia. The medical device may be used to maintain the indoor heat balance of a body of the individual, with or without the use of behavioral thermoregulation on the behalf of the user.

In some forms, the medical device may connect a closed loop control system of the dual input thermostat with a thermoregulatory closed loop control system of a body of the individual to create a third interactive loop between the two. The device may do this, while still allowing the closed loop control system and the thermoregulatory closed loop control system to control autonomously.

According to another aspect of the invention, a method is disclosed of operating the medical device of the type described above. According to the method, the toe temperature is measured using the remote toe temperature sensor and the air temperature is measured using the air temperature sensor. The toe temperature from the remote toe temperature sensor and the air temperature using the air temperature sensor are both provided to the dual input thermostat. Based on these provided temperatures, the set point temperature of the HVAC system is adjusted relative to the air temperature inversely and proportionately based on the toe temperature provided by the remote toe temperature sensor. As noted above, the set point temperature is bounded by the range of temperature operation for the HVAC system delimited by the high limit room temperature setting and low limit room temperature setting of the dual input thermostat.

In some forms, the step of providing the toe temperature from the remote toe temperature sensor to the dual input thermostat may involve wirelessly transmitting the toe temperature to the dual input thermostat.

In some forms, the method may further include the steps of receiving an input from the control(s) to adjust at least one of the high limit room temperature setting and low limit room temperature setting and of adjusting the range of temperature operation for the HVAC system.

In some forms, the step of adjusting the set point temperature of the HVAC system relative to the air temperature may occur in response to a change in the toe temperature and the step of adjusting may be time delayed to dampen the response to the toe temperature sensor. In this way, temporary and fleeting changes in temperature may not be immediately translated in alterations the set point temperature.

The foregoing and other objects and advantages of the invention will appear in the detailed description which follows. In the description, reference is made to the accompanying drawings which illustrate a preferred embodiment of the invention.

### BRIEF DESCRIPTION OF THE DRAWINGS

FIG. 1 is a flow diagram showing the basic operation of a room thermostat.

FIG. 2 is a flow diagram showing the basic operation of the thermoregulatory system.

FIG. 3 is a flow diagram of this invention showing the interaction between the room thermostat and the thermoregulatory system.

FIG. 4 is an illustration of a human body showing the distribution of temperatures in the body's core and shell.

FIG. 5 is a front view of the preferred embodiment of the dual input thermostat and a wirelessly connected remote toe temperature sensor.

### DETAILED DESCRIPTION OF THE PREFERRED EMBODIMENT

The detailed description that follows includes two sections. In the first section, a new, better understanding of vasoconstriction and its relationship to age-related diseases is presented. In the second section, a medical device involving a dual input thermostat and a body sensor on an extremity (specifically, a toe temperature sensor) is provided as a way to improve thermoregulation to prevent age-related diseases induced by long-term cool indoor temperatures.

Current medical science generally believes that local vasoconstriction, like reflex vasoconstriction, is also a homeostatic thermoregulatory response that regulates temperature. A more comprehensive understanding of this response which is explained here, shows that local vasoconstriction by itself, cannot regulate. Local vasoconstriction is also shown to be a positive feedback defense mechanism which may become harmful with excessive indoor use. This newly recognized knowledge of local vasoconstriction is believed to be novel and is thus first-presented. Then, subsequently, with an appreciation of this knowledge in mind, a device will be disclosed for assisting an individual with maintaining thermoregulation.

### Locally-mediated Vasoconstriction

Local cooling of the skin engages local constrictor mechanisms, independent of reflex vasoconstriction activity. Local vasoconstriction is controlled with positive feedback. Positive feedback control mechanisms accelerate or enhance the sensed change and attempt to push levels out of normal ranges, that is, a drop in sensed local skin temperature increases the intensity of local vasoconstriction which drops the local skin temperature even further. Activated local vasoconstriction is not configured to regulate or warm its sensed local skin temperature. This response is only configured to defend core body temperature, and for this purpose positive feedback is necessary, that is, a decrease in local skin temperature leads to further cooling of the local skin, which reduces local heat loss indirectly warming the core. Because positive feedback is self-reinforcing, this indicates that local vasoconstriction cannot regulate and is only a defense mechanism. When responding to an indoor whole body cold stimulus, most thermoregulatory responses occur in an orderly progression of need. If this is also true here, local vasoconstriction would activate as the intensity of reflex vasoconstriction becomes maximized and further defense of core body temperature is needed. A study of cutaneous cooling-sensitive receptor TRPM8 in mice showed an activation skin temperature of 28.4 degrees C. The activation skin temperature in humans and its receptor is unknown. Another study reports that maximal vasoconstriction and minimal blood flow occur when the local skin temperature is about 15 degrees C. Local vasoconstriction can be more intense and can reduce local blood flow and local skin temperatures to a far greater extent than reflex vasoconstriction, as evidenced by the lower skin temperature when maximized.

It is likely that the body did not intend for local vasoconstriction to be used for the normal everyday maintenance of core body temperature. Both responses defend core body temperature by reducing heat loss, but only reflex vasoconstriction always does it safely. To decrease heat loss a given amount, reflex vasoconstriction uses a mild constriction to produce a small drop in skin temperature safely spread over most of the periphery. For an equal decrease in heat loss, local vasoconstriction involves an intense constriction producing a large drop in local skin temperature in one much smaller location. Over time, this intensive constriction can adversely affect the blood circulation at this location.

The negative feedback of reflex vasoconstriction provides for the continuous regulation of heat balance, and is always beneficial. When a person is outdoors in cold climates, the positive feedback of local vasoconstriction defends core body temperature to prevent hypothermia. Local vasoconstriction and other body defense mechanisms like inflammation and fever, while beneficial, can eventually become harmful when their use becomes excessive. Local vasoconstriction may be considered excessive when it is frequently needed, while indoors, to defend core body temperature. By its very nature, if a positive feedback defense mechanism becomes chronic, it is likely to eventually cause progressive harm. Degree and duration of positive feedback vasoconstriction determines the likelihood of harm. However, when the body needs local vasoconstriction to avoid hypothermia, indoors or outdoors, the near term benefits become more important than any long term consequences. As a positive feedback response, local vasoconstriction is inherently progressive and needs to be constrained.

### Vasoconstrictive Interaction

The body's physiological ability to both produce and conserve heat has its limits, especially in the elderly. A plausible mechanism to explain how negative feedback reflex vasoconstriction interacts with positive feedback local vasoconstriction, is as follows: In those elderly that are most at risk, an unperceived indoor whole-body cold stimulus of sufficient degree or duration, can drive the reflex vasoconstriction to its maximum intensity, reaching a limit where it is unable to have any further effect. Regulation of heat balance is temporarily lost. This increases the gradient between core and peripheral body temperatures. The lower legs are most distal to the core and typically have the highest gradient and coldest skin temperature. When a location on a lower leg drops below the local vasoconstriction activation temperature, positive feedback local vasoconstriction will be activated at that site. Positive feedback mechanisms are progressive by nature and continue to enhance the sensed change. If left unchecked, they will ultimately destroy themselves. Positive feedback local vasoconstriction is kept in check or constrained by the actions of negative feedback reflex vasoconstriction. The impact of activated local vasoconstriction is immediate as constriction drives the local skin temperature ever colder. This local conservation of heat brings forth a very slight warmth in the rest of the body. Reflex vasoconstriction responds to this warmth by decreasing from its maximum intensity, until it regains regulation of heat balance. The interplay of reflex and local control mechanisms is an intricate balancing act. Local vasoconstriction attempts to drive the local skin temperature colder at the same time reflex vasoconstriction varies the warmth at the local site's outer or leading edge to constrain the cold skin surface area. Reflex vasoconstriction is in control of the cold site's leading edge allowing the surface area to expand if needed or adding warmth to force its retreat. This constraint of local vasoconstriction is needed for reflex vasoconstriction to maintain regulation of heat balance. Reflex and local vasoconstriction interacting together are able to conserve more heat than maximized reflex vasoconstriction can conserve by itself, thereby preventing hypothermia. This interaction could occur occasionally or often, and the cooler extremity skin temperatures may never be recognized. Excessive indoor local vasoconstriction may also be called chronic and needs to be better understood. It needs to be recognized by the individual, or diagnosed by a doctor, and prevented.

Once activated, positive feedback local vasoconstriction is not easily deactivated. Local vasoconstriction cannot regulate, warm or deactivate itself. To transition back to where heat balance is maintained by reflex vasoconstriction alone, it is necessary to deactivate local vasoconstriction. This requires a decrease in cold stimulus to bring about a less intensive reflex vasoconstriction. Without awareness to decrease the cold stimulus, de-activation may involve an inadvertent increase in warmth from taking a bath, going to bed, or a change of environment. When homeostatic heat balance favors a warmer skin temperature, less intensive reflex vasoconstriction will provide a stronger blood supply to the outer edges of the cold skin area. This warming occurs from the outer edges inward toward the center or from the proximal to the distal end of the extremity, until local vasoconstriction gradually disappears, stopping the vasoconstrictive interaction.

### Age-Induced Cold Stress

The term cold stress as used here is an unrecognized mild cold stress occurring in an older person's everyday indoor environment. One effect of aging is an ongoing natural progression of cold stress. It is known that reflex vasoconstriction is markedly impaired in healthy aged skin. Age-impaired reflex vasoconstriction leads to higher skin blood flows during cold exposure and results in an increase in heat loss that progresses with age. Over decades as we age, the body also declines in its capacity to produce heat. Decreasing metabolic heat production in the elderly is generally associated with a lower basal metabolic rate, less lean body mass, less physical activity, and a lower intake of food. Thus, as a person's age progresses progressively more heat is being lost while progressively less heat is being produced. This age-induced cold stress, along with a diminished perception of the cold, creates ever greater use of vasoconstriction to maintain heat balance. This makes vasoconstriction progressive throughout later life. As reflex vasoconstriction loses its ability to constrict with age, progressively more local vasoconstriction will be required to conserve additional heat and maintain heat balance. An older person who frequently has cold hands or feet while indoors, may be unaware that he or she is experiencing age-induced cold stress.

Under an indoor whole-body cold stress, reflex vasoconstriction maintains the body's heat balance and prevents the need for local vasoconstriction until it reaches its maximum intensity and can no longer affect either. After this, local vasoconstriction activates to conserve additional heat which prevents hypothermia. Unlike reflex vasoconstriction which becomes impaired with age, the magnitude of local vasoconstriction is known to remain unaffected with age. That allows this positive feedback mechanism to retain its powerful defense against hypothermia. This powerful defense is the reason indoor hypothermia is extremely rare. As a person ages, normal healthy reflex vasoconstriction is diminished and progressively more potentially harmful local vasoconstriction is needed to prevent hypothermia. Hypothermia would appear to be prevented until local vasoconstriction also becomes maximized and it too can no longer conserve additional heat.

Age has always been thought to be an uncontrollable risk factor for most age related diseases. This is not necessarily true. The fundamental biological mechanisms that underlie the aging process are unknown, but an age-related disease should have an age-related cause. It is hypothesized herein that age-induced cold stress, combined with cold stress from the risk factors for age-related disease, causes many of the age-related diseases. If and when this can be shown to be true, age can be compensated for with warmth. Ample warmth can prevent local vasoconstriction, and eliminate age as a risk factor for all cold-induced age-related diseases.

### Risk Factors for Age-Related Disease

The risk factors for many different age-related diseases are often the same or similar. They typically include advanced age, diabetes, sedentary lifestyle, obesity, smoking, malnutrition, excessive alcohol consumption, and hypertension. All of these have one thing in common. Each of these risk factors (except hypertension) either decreases metabolic heat production or increases heat loss, leading to cold-induced vasoconstriction. From this, it would be intuitive to conclude that cold-induced vasoconstriction is a factor in the occurrence of age-related disease.

Persons with decreased heat production include the aged as mentioned above and also those with a disease that inhibits metabolic heat production, like diabetes or hypothyroidism. Those with a sedentary lifestyle do not produce as much heat as those who are physically active. Malnutrition can adversely affect metabolism and also decrease the body's heat production. Persons with increased heat loss would include the aged because of age-impaired reflex vasoconstriction. Excessive alcohol consumption is known to cause vasodilation, leading to higher skin blood flows which will also increase heat loss. Increased heat loss also occurs in the obese where weight gain increases skin surface area. Heat loss is proportional to skin surface area. An increase in surface area increases total heat loss without an equal corresponding increase in total heat production. The major heat producing organs, such as the liver, heart, and brain do not get larger or produce more heat with obesity.

Research shows that those who carry their excess weight in the abdomen (apple-shaped) are more at risk for type 2 diabetes and cardiovascular disease, than those who carry their excess weight in their hips and buttocks (pear-shaped). This phenomenon is not well understood. A plausible explanation here is because obesity increases skin surface area, the fat locations coincide with the locations of additional heat loss. When a person carries their obesity in their hips and buttocks, this added heat loss occurs in a non-vital location. When a person carries their obesity in their abdomen, this additional heat loss is located adjacent to temperature sensitive vital organs ill-equipped to defend against this added cold stress.

Smoking also increases cold stress. Smoke contains carbon monoxide which attaches itself to the hemoglobin in red blood cells much more easily than oxygen does, effectively reducing the oxygen-carrying capacity of the blood. This deprives tissues and organs of oxygen and reduces the amount of oxygen available for energy or metabolic heat production. This decrease in heat production will induce an equal corresponding increase in vasoconstriction to maintain the body's heat balance.

Increased vasoconstriction is known to elevate blood pressure. Numerous surveys and studies have documented the inverse correlation between temperature and blood pressure. This suggests that cold-induced hypertension, is not a risk factor itself, but an effect of all the other risk factors. Aging is also known to raise blood pressure. A plausible explanation for this is because of age-induced cold stress, a progressive increase in age induces an equally progressive increase in vasoconstriction, which is known to raise blood pressure. Cold-induced hypertension may be best treated by using a warming means to decrease the cold stress from these other risk factors, thereby decreasing vasoconstriction which lowers the blood pressure.

Each age-related disease risk factor, including age, can induce a cold stress on the body. Multiple risk factors combine with age to maximize cold stress and speed its progression. Because age and the other risk factors all have cold stress in common, each risk factor can be weighted against the others, to approximate the contribution of each. Age itself may be a major or just a minor contributor to the total cold stress. If age is the only risk factor inducing cold stress, age-related disease may not occur for a very long time, if ever. If age is one of many presented risk factors inducing cold stress, the total cold stress is higher and age-related disease is much more likely. Age-induced cold stress progression, if not stopped, will keep on progressing at the rate of chronological age, as a minimum. This age-induced progression of cold stress is also a plausible explanation why so many age-related diseases become chronic diseases.

Taking cold stress full circle, for a person outdoors in cold weather, it is known that the risk for frostbite, non-freezing cold injuries, or hypothermia is increased in those that have predisposing health conditions. These predisposing health conditions include cardiovascular disease, diabetes, hypothyroidism, hypertension, or advanced age. The predisposing health conditions for hypothermia (i.e. cold stress) are all age-related diseases. This suggests a two-way cause/effect relationship between cold stress and age related disease.

### Inflammatory Pathway

Cumulative exposure to decreased temperature is associated with an increase in inflammation marker levels among elderly men. Chronic inflammation has also been linked to the biological aging process. This may be because of age-induced cold stress. Evidence indicates that chronic inflammation with advancing age can precede several diseases. This suggests that inflammation may be a pathway or part of an intermediate process between an initiating cold stimulus and age-related disease.

Risk factors associated with chronic inflammation include: advanced age, obesity, diabetes, smoking, and poor nutrition. These risk factors are the same as the risk factors for age-related diseases. All of these risk factors decrease metabolic heat production or increase heat loss. This suggests that cold stress is a factor in the occurrence of cold-induced inflammation and may provide a pathway to cold-induced age-related disease.

It is evident from extensive observations and experiments within the last few decades that most chronic diseases are preceded by a chronic low level of inflammation. The major chronic diseases associated with inflammation and aging are cancer, cardiovascular disease, diabetes, pulmonary disease, and neurological disease.

### Atherosclerosis

The relationships between cold temperatures and cardio-respiratory mortality in the elderly are well documented. Most of the excess mortality is due to respiratory cardiac and cerebrovascular disease. The strong indirect epidemiological evidence coupling cold climate to mortality may be related to indoor rather than outdoor climate conditions coupled with a plethora of factors including health status and aging-related deterioration in physiological and behavioral thermoregulation.

In those most susceptible to an indoor cold stress, a cold periphery caused by excessive cutaneous vasoconstriction, may eventually challenge the body's ability to maintain a healthy core body temperature. It is known that maintenance of a stable core temperature within very narrow limits is a basic need of man. The body's core is generally thought to maintain a homogenous temperature. While this is typically true, it may not be the case when the core is severely challenged by cold stress. A hypothetical non-peripheral defense mechanism to protect against core cold stress would need to increase the core's heat production, decrease its heat loss, or both. Arterial blood supplies to capillary beds of vital organs are very sensitive to temperatures below 37 degrees C. Skin, muscle, bone, and some fat tissues in the core are non-vital and like the extremities would not incur harm at lower temperatures. It would be logical and advantageous for a core defense to only defend the vital temperatures, maintaining 37 degrees C. only where it is needed most. This hypothetical core defense mechanism would create a thermal heterogeneity within the core that could be recognized when under a cold stress. Thermal heterogeneity in the body's core has already been discovered, measured, and written about in numerous research papers. It has just not been recognized as a core temperature defense mechanism.

Cold exposure has been shown to promote atherosclerotic plaque growth. Atherosclerosis is an inflammatory disease. Inflammation is known for its ability to produce heat and plays a role in all stages of atherosclerosis. It is well known and widely accepted that inflammation can be both beneficial and harmful. It should therefore not be irrational to conclude that atherosclerotic inflammation can also be both beneficial and harmful.

My hypothesis is: “Atherosclerosis is a beneficial defense mechanism, producing heat to locally warm arterial blood flows.” Metabolic heat produced by macrophages accounts for the local inflammatory reaction present in the plaque. Plaque can release heat directly to the blood and this heat has been correlated positively with inflammatory cell density, with both increasing as the atherosclerosis progresses. Elevated plaque temperature, also known as thermal heterogeneity, is measured with various devices and is studied as a means of predicting future cardiac events. Many of these studies recognize the arterial blood supply as cooling harmful plaque. They should be recognizing the beneficial plaque as warming the arterial blood supply. Plaque advantageously develops at arterial locations where blood flow is turbulent. This turbulence is very beneficial because it enhances heat transfer from the plaque to the blood.

Atherosclerotic plaque may extend our lives for years by preventing dangerously low arterial blood temperatures to vital organs in the core. This is before eventually harming us, as we overuse the good it does. When cold stress is allowed to progress with age, inflammation and plaque are likely to progress as well. When the body needs atherosclerotic inflammation to defend against low arterial blood temperatures, the near term benefits are more important than any long term consequences. Understanding why atherosclerosis occurs enables us to use a warming means to prevent its need.

Any local or systemic disease that is age-related, progressive, thought to be caused by poor or impaired blood circulation, or associated with cold weather, may be cold-induced and, if so, would be treatable with warmth. There are perhaps hundreds of named diseases that could fit this description. If we can prove that cold stress causes age-related disease, it would mean that these diseases are likely reversible, over time, with removal of the cold stress. It also means that these diseases could be prevented, by simply keeping warm. The therapeutic room thermostat can provide this drug free warmth to an elderly or diseased person when their behavioral regulation or their reflex vasoconstriction is no longer adequate.

The goal is successful aging, where people are able to live long enough to die from old age, rather than dying prematurely from an age-related disease.

### Thermostat Operation

Thermostats have always maintained comfort. The newly disclosed thermostat looks beyond comfort to maintain health. The therapeutic room thermostat remotely senses a body's skin surface temperature on or near the toes and uses toe temperature to modify the air temperature of that individual's environment, to keep thermoregulation within their effective range of reflex-mediated vasoconstriction. The therapeutic room thermostat is intended to replace existing room thermostats in private hospital rooms and intensive care units, private nursing home rooms, single person apartments, and private offices. This personalized thermostat is ideally suited for those with a poor or impaired perception of temperature, such as diabetics, and the elderly. For them, the therapeutic room thermostat constantly adjusts to their thermoregulatory needs, when they are not able to do so themselves. The therapeutic room thermostat is a medical device including both a dual input thermostat and a remote toe temperature sensor.

The dual input thermostat may receive inputs from both an internal room temperature sensor and an external remote toe temperature sensor. The toe temperature is used to reset the room temperature within low limit and high limit settings. The low limit setting can be set at the preferable setting of a conventional room thermostat. This low limit setting may also serve as the default setting whenever toe temperature is not sensed, such as when the person leaves the room. The high limit setting represents the higher temperature needed, at times, to keep reflex vasoconstriction from becoming maximized. This high limit setting would be expected to be increased with age and poor health. The dual input thermostat may be a smart thermostat capable of internet communication. It could also be a learning thermostat that would anticipate time of day or circadian rhythm influences on the body's cold stress.

The remote toe temperature sensor provides a skin temperature input to the dual input thermostat. This sensor also provides an input to a warning light to indicate a low toe temperature. Skin surface temperature varies directly with cutaneous blood flow. As vasoconstriction decreases the cutaneous blood flow, the skin temperature decreases as well. A warmer skin surface temperature prevents positive feedback vasoconstriction and keeps thermoregulation within the effective range of negative feedback vasoconstriction. For most accurate use, the toe temperature sensor is not to be used on a skin surface that shows any signs of inflammation. Inflammation is known to produce warmth and this warmth will conflict with warmth provided by this therapeutic room thermostat. A contralateral location which is healthy should be chosen for the toe temperature sensor. When warmth is maintained first by the therapeutic room thermostat at a non-inflamed site, warmth from inflammation may be inhibited.

The oldest and simplest method of controlling temperature is open loop control. Examples of open loop control are: throwing another log on the fire, opening or closing a hand valve on a radiator, and adjusting the damper on a stove or fireplace. Open loop control is not self-regulating.

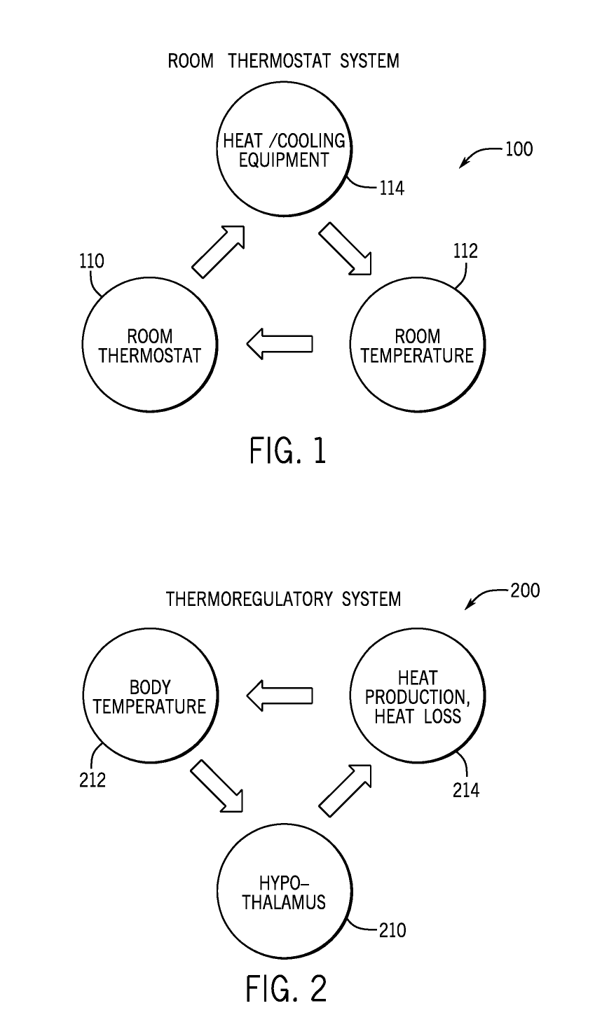

In the late 19th century, the invention of the electric room thermostat created an automatic temperature control system. Referring to FIG. 1, a flow diagram of a thermostat's operation is provided in a room thermostat system 100. The room thermostat system 100 maintains a constant room temperature as determined by its set-point. A thermostat 110 senses room temperature 112 and compares this to its set-point. If the room is colder than the set-point, for example, the thermostat 110 will turn on the heat using connected heating and/or cooling equipment 114 (otherwise known as an HVAC system). As the room temperature 112 increases, there is a feedback effect on the thermostat 110 that warms its sensor. When the room warms to the set-point, the thermostat 110 turns off the heat. This operation, self-regulating or automatic, is a closed loop control system which basically has not changed in over a hundred years.

The human body is essentially a constant temperature system. Its internal temperature is maintained at 37 degrees C. by the thermoregulatory system. Referring to FIG. 2 which is a flow diagram of basic control for a thermoregulatory system 200. The thermoregulatory center is located in a part of the brain known as the hypothalamus 210. The hypothalamus 210 receives messages from thermal receptors located throughout the body to indicate body temperature 212, and determines the need to produce or conserve body heat, or to increase heat loss using body temperature regulatory functions 214 based on this feedback. The body's thermoregulatory control system is self-regulating and is also a closed loop control system.

The room thermostat is a constant temperature system, typically 21 degrees C. The human body is a constant temperature system, typically 37 degrees C. When a human enters a room, there is a potential conflict. Each control system maintains its own respective temperature regardless of the needs of the other. There is no method or means of communication to enable the two individual control systems to work together to maintain the body's heat balance and therefore its health.

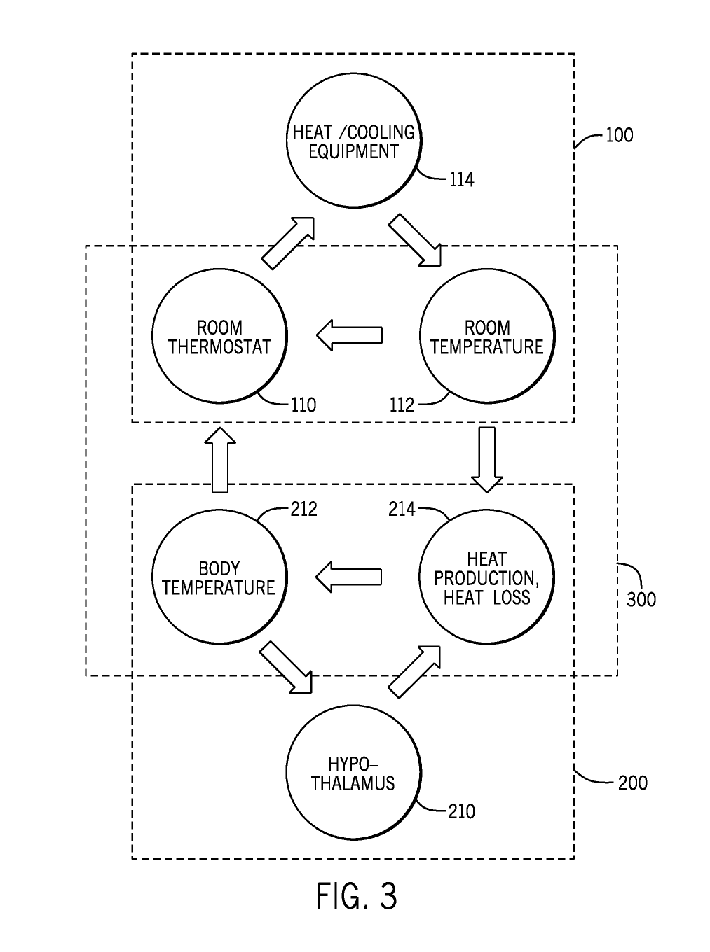

Referring to FIG. 3, this invention joins the room thermostat's closed loop controlled system 100 with the body's thermoregulatory closed loop controlling system 200, enabling both to work together. This invention joins the two closed loops to create a third interactive loop 300 while still allowing the two closed loops to control autonomously. As an example, an imbalance which causes a decrease in body temperature 212 will raise the set-point of the room thermostat 110 and increase room temperature 112. This decreases the body's heat loss at 214 thereby increasing the body temperature 212 to create a new balanced condition. In this system, the thermoregulatory loop 200 acts as the controlling loop while the room thermostat loop 100 becomes the controlled loop.

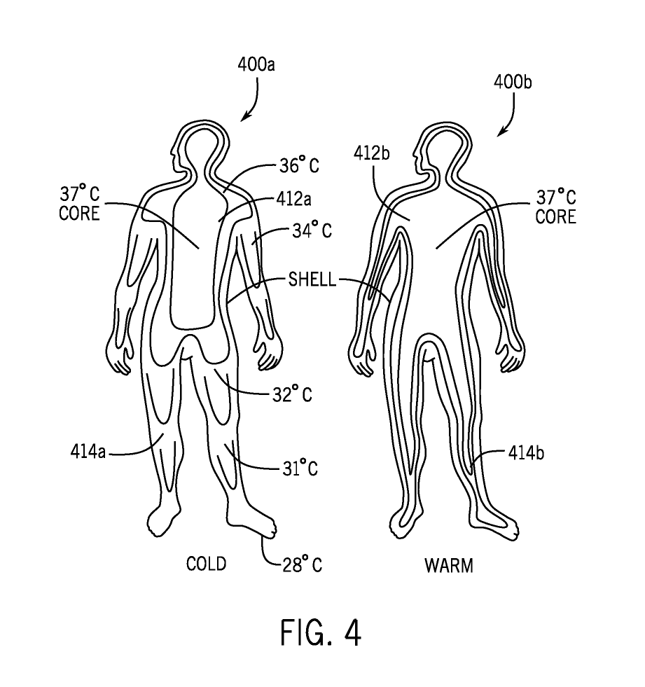

Referring now to FIG. 4, the distribution of temperatures in the body's core and shell is illustrated in two different scenarios—one in which the extremities of the shell exhibit a significant gradient from the core (that is, in body 400a) and one in which the extremities do not exhibit a significant graditent (that is, in body 400b). The bodies 400a and 400b are divided into a warm internal core 412a, 412b and a cooler outer shell 414a, 414b, respectively. The temperature of the shell 414a, 414b is strongly influenced by the environment and the body's need to conserve or dissipate heat. The internal core body temperature of the vital organs inside the head and trunk is closely regulated at 37 degrees C. As cold-induced vasoconstriction reduces the skin blood flow, the affected skin becomes cooler. The underlying tissues become cooler as they lose heat by conduction to the cool overlying skin. These underlying tissues, which in the heat were part of the core, now become part of the shell. By preventing local vasoconstriction, the medical device of this system tends to minimize the shell while maximizing the core.

In the disclosed system, the controlled room temperature has an inverse relationship with the skin temperature. Suppose the room temperature low limit is set at 72 degrees F. and the high limit is set at 76 degrees F. We want to use warmth to keep the toe temperature above 86 degrees F. (30 degrees C.) and any toe temperature above 93.2 degrees F. (34 degrees C.) does not need any additional warmth. As an example, with a toe temperature of 93.2 degrees F. (34 degrees C.) or above, the room temperature will maintain its low limit of 72 degrees F. With a toe temperature of 86 degrees F. (30 degrees C.) or below, the room temperature will maintain its high limit of 76 degrees F. When the toe is between 30 and 34 degrees C. (between 86 and 93.2 degrees F.), the room temperature will vary proportionately and inversely with the toe temperature, such that a 32 degree C. (89.6 degrees F.) toe temperature will maintain a room temperature of 74 degrees F. These limits and toe temperatures can be adjusted. The toes are most distal to the core and typically have the coldest skin temperature on the body. If the toe temperature is above the local vasoconstriction activation temperature, local vasoconstriction caused by indoor whole-body cooling, is not normally present in the body. The local vasoconstriction activation temperature is presently unknown and needs to be determined with further research. A healthy indoor environment exists when the body's heat balance is maintained within the dynamic range of reflex vasoconstriction, preventing the need for local vasoconstriction. The therapeutic thermostat has the ability to provide this healthy indoor environment when the body's thermoregulatory system is no longer able to do so.

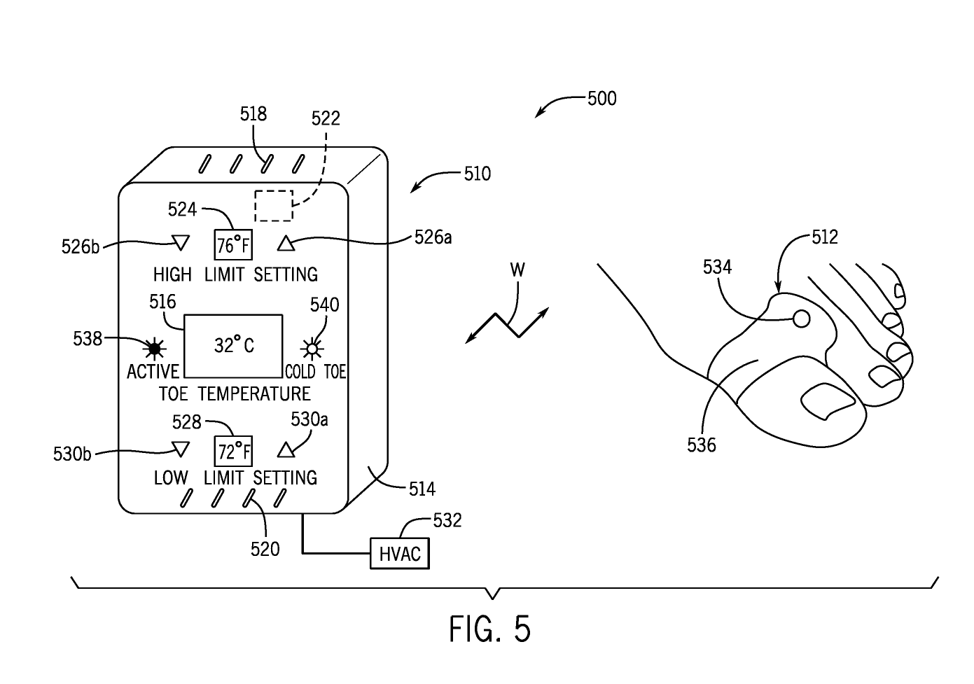

Referring to FIG. 5, the therapeutic room thermostat or medical device 500 includes both a dual input thermostat 510 and a remote toe temperature sensor 512. The preferred embodiment of the dual input thermostat 510 comprises components on the surface of the housing 514 which are shown, and internal components which are described but not shown. The toe temperature display 516 indicates the actual toe temperature. Actual room temperature is of limited interest in this system because toe temperature is the vital indicator. While room temperature may be controlled, it is the effect on control of toe temperature that is of most interest because it is the aim of the device 500 to result in increased temperature at the extremities of the body. An inlet grille 520 and an outlet grille 518 allow for a natural flow of room air thru the thermostat 510 where it internally flows over an air temperature sensor 522 (schematically depicted by box 522) for the room. The internal room temperature sensor 522 provides an input of the air temperature to the dual input thermostat 510. The high limit setting display 524 indicates the desired high limit setting which is raised with adjustment controls 526a and lowered with adjustment control 526b. The low-limit setting display 528 indicates the desired low limit setting which is raised with adjustment control 530a and lowered with adjustment control 530b. Although these controls are illustrated as being four separate buttons, it will be appreciated that those controls may take various forms and be combined with one another (for example in a depressible, rotatable dial) or may be located off of the thermostat 510 (for example, it may be wireless controlled by a remote or smartphone application). For those individuals with a better perception of temperature, the high and low limit settings are intended to be adjusted close to their high and low comfort thresholds. Ideally, the therapeutic room thermostat 510 will instruct a connected HVAC system 532 (illustrated schematically) to provide the temperature required to keep an aged or diabetic person closer to their thermoneutral zone. This is the range of ambient temperatures in which minimal metabolic energy is expended for thermoregulation. The high and low limit settings define the extremes of this temperature range which is intended to be beneficial to health.

When the room occupant has a toe temperature of 34 degrees C. or above as sensed by the remote toe sensor 512, the therapeutic room thermostat 510 controls normally at the low limit setting. A sudden change in toe temperature, perhaps caused by the occupant getting out of bed and walking barefoot to the bathroom, could quickly change the room temperature and is undesirable. For this reason, the dual input thermostat 510 may have a time delay incorporated to slow any room temperature change when it is floating between both limit settings. Whenever the room temperature is at either of the limit settings, this time delay is inactive.

In order to operate, the dual input thermostat 510 also receives a second input from the remote toe temperature sensor 512. This remote toe temperature sensor 512 should be compatible with the dual input thermostat 510 and would preferably be wireless to transmit a temperature measured at the skin surface of the toe of the individual wearing the sensor 512 to the thermostat 510 (as generally denoted by wireless communication signal W), however the signal could also be provided in a hardwired fashion. The remote toe temperature sensor 512 is preferably comfortable while wearing shoes and walking. One embodiment would locate the sensing probe 534 the remote toe temperature sensor 512 within a molded soft silicone spacer 536 fitted between the great toe and its adjoining toe, as shown in FIG. 5. This embodiment of the remote toe temperature sensor 512 might include a metal housing for proximal contact with the skin in which a tiny battery powered temperature sensor probe and transmitter are received (combined schematically at 534 in the illustrated figure). This probe and transmitter 534 would measure the temperature and transmit a signal, indicating the toe skin temperature, over a maximum distance of perhaps thirty feet. The toe temperature sensor 534 is non-disposable and fitted inside a pocket of the soft silicone spacer 536 which is disposable. Whenever the dual input thermostat 510 is receiving a temperature signal from the remote toe temperature sensor 512, toe temperature active light 538 will be lit to indicate that the dual input thermostat 510 is actively responding to the toe temperature input. When the toe temperature active light 538 is not lit, this means that the dual input thermostat 510 is not receiving a toe temperature input. When this occurs, the dual input thermostat 510 will control at the low limit set-point continuously. An unhealthy situation could arise if the high limit setting was adjusted too low to be able to maintain at least 30 degrees C. at the toe. When this condition exists for a predetermined time period, a cold toe temperature light 540 would light up. This would indicate that the high limit setting needs to be raised to adequately warm the toe. The cold toe temperature light 540 could indicate in red, while the toe temperature active light 538 indicates in green to distinguish them from each other at a distance. As an alternative to individual lights, a thermostat background lighting means could change from off to green or to red under the described conditions.

The therapeutic room thermostat as previously described is suitable for one individual in a private room. However, many hospitals and nursing homes use semiprivate rooms designed for two people with one thermostat. The therapeutic thermostat for two is another embodiment of this invention designed for two individuals sharing one room and one room thermostat. Both individuals wear a remote toe temperature sensor 512 and the therapeutic thermostat 510 for two receives both inputs and selects the coldest toe temperature of the two. Room temperatures in between both limit set-points are determined by the coldest toe. While comfort can never be assured when two share a single thermostat, an environment without cold stress can be assured for both individuals with this embodiment.

Presently, thermostats in health facilities are generally adjusted by the facility's staff. Comfort levels between a young, healthy staff and the elderly patients differ widely. Knowing now that cold stress can cause harm to elderly patients, the patient's health should take priority over the staff comfort level. The therapeutic thermostat is one way of assuring this healthy environment.

During typical use, the dual input thermostat 510 provides instructions to the HVAC system 532 to control the air temperature to a set point. In some instances, the volume of the air for which the air temperature being regulated by the HVAC system 532 may be defined as being within a particular operational zone of the building such as, for example, a room. A particular house or structure may contain multiple operational zones (for example, there may be multiple patient rooms all separately controlled within a hospital) or may only contain a single operational zone (for example, the entirety of a relatively small house). As can be understood from the above description, the use of a toe temperature sensor 512 with the dual input thermostat 510 in aggregate can be used as a medical device or therapeutic device to maintain thermoregulation within an effective range of reflex-mediated vasoconstriction for an individual when the individual is subjected to a mild indoor cold stress. However, it is also contemplated that the dual input thermostat 510 could act as a standard thermostat if the toe temperature sensor 512 is not being worn by an individual or if the dual input thermostat 510 is otherwise set to operate to drive the HVAC system 532 independently to reach a set point without regards to the temperature of the toe temperature sensor 512.

However, in the preferred usage condition, the remote toe temperature sensor 512 is attached on or near a toe of an individual to provide a toe temperature. This toe temperature is provided to the dual input thermostat 510 as one of multiple inputs for operation. The other input, in most circumstances, is from the air temperature sensor 522 which measures the air temperature in the operational zone. The air temperature sensor 522 should be within the particular operational zone of the HVAC system 532 and, in many instances is within or supported by the housing 514 of dual input thermostat 510. However, it is also contemplated that the air temperature sensor 522 could be remote from the thermostat 510 with the air temperature sensor 522 being positioned in the operational zone and the thermostat 510 being positioned outside of the operational zone. It should also be appreciated that the term “dual input thermostat” is used to describe that the thermostat receives at least two inputs including an air temperature and a toe temperature, but other inputs in excess of two inputs may be provided. Accordingly, “dual” should be understood as meaning two or more inputs. To give an example, multiple toe temperature sensors or multiple air temperature sensors could be connected with a single thermostat and the data aggregated to operate the thermostat and HVAC system. In yet another version, a single thermostat may operate multiple operational zones (each having a respective air temperature sensor); in this instance, more than two inputs would be available to the thermostat.

In any event, with the toe temperature and the air temperature provided to the thermostat 510, the thermostat 510 adjusts the set point temperature of the HVAC system 532 relative to the air temperature. This adjustment occurs inversely and proportionately based on the toe temperature provided by the remote toe temperature sensor 512 and within the range of temperature operation for the HVAC system 532 delimited by the high limit room temperature setting and low limit room temperature setting of the dual input thermostat 510. For example, if the toe temperature provided to the thermostat 510 is low (for example, below a threshold temperature described above indicating a situation in which the extremities are cold and need warming), then the set point on the thermostat 510 is increased. Similarly, if the toe temperature provided to the thermostat is high, then the thermostat 510 may be adjusted to a lower temperature. However, in any event, the set point of thermostat 510 will not be adjusted outside of the range of operation selected by the controls 526a, 526b, 530a, and 530b.

It is contemplated that certain conditions may occasionally result in abrupt temperature changes in the toe temperature sensor 512 and, accordingly, there may be some time delay in response by the thermostat 510 before adjusting the set point of the HVAC system 532. Among other things, this time delay can help to dampen the cycling of the HVAC system 532, if for example, an individual wearing the toe temperature sensor 512 removes a sock or footwear or the toe temperature sensor 512 is temporarily removed. Similarly, if the toe temperature sensor 512 provides an erratic false reading, then having some dampening or time delay may be helpful in eliminating static or anomalies from the toe temperature history.

In the event that the individual wishes to manually set the high or low temperature set points, these adjustments can be made manually using the controls 526a, 526b, 530a, and 530b on the thermostat 510.

It is contemplated that, in some forms, the toe temperature sensor 512 might not only provide toe temperature measurements, but may also provide spatial information about the location of the toe temperature sensor 512. For example, based on signal strength or relative signal strength, a system may be built that detects the location of the toe temperature sensor 512 relative to a plurality of receiving locations (for example, receiving stations which may be located at separate thermostats or at different locations in a building). Based on this information, a location of the individual may be determined, such as which one of a plurality of operating zones an individual is positioned in. Such information may be used to selectively drive the appropriate HVAC system or portion thereof to efficiently adjust the temperature in the zone the individual with the toe temperature sensor is located in.

Still yet, it is contemplated that in some embodiments that device comprising the toe temperature sensor and/or the thermostat may be connected to other devices (internally or externally) to provide temperature information about the toe of the individual. For example, the thermostat or a connected secondary device may record a temperature history of the toe of the individual. This information may be collected for use by the individual or health providers in establishing the individual's temperature profile. Still yet, the toe temperature could be provided via the internet or other communications connections to a central server that monitors the temperature remotely. Should an toe temperature persistently remain below a threshold temperature (or be within a predetermined range incrementally above room temperature but below an acceptable range for toe temperature in a healthy body), an alert may be provided that the individual's toe temperature is outside of the acceptable range. Such information could permit caretakers or hospital staff, whether locally or remotely located, to check in on the individual or provide preventative care based on the observed temperature condition.

Accordingly, a device is provided that permits adjustment of a temperature in a room based on provided temperature information from a sensor disposed on or near the toe of the user. This information can be used to proactively adjust temperature to reduce coldness in the extremities which can offer various long term health benefits. This device can help to make identify conditions and make appropriate adjustments when the individual themselves may otherwise not feel cold or take the appropriate steps to warm their extremities. By use of this device, an individual can passively make beneficial temperature adjustments to warm their body and maintain good health without having to manually monitor their temperature conditions.

A preferred embodiment of the invention has been described in considerable detail. Many modifications and variations to the preferred embodiment described will be apparent to a person of ordinary skill in the art. Therefore, the invention should not be limited to the embodiment described.

I claim:

1. A medical device to maintain thermoregulation within an effective range of reflex-mediated vasoconstriction for an individual when the individual is subjected to a mild indoor cold stress, said medical device comprising:
a remote toe temperature sensor for placement on or near a toe of the individual, the remote toe temperature sensor providing a toe temperature; and
a dual input thermostat adapted for connection to an HVAC system for adjusting an air temperature in an operational zone to a set point temperature and further adapted for connection to the remote toe temperature sensor to receive the toe temperature, the dual input thermostat including at least one control for the adjustment of a high limit room temperature setting and a low limit room temperature setting to delimit a range of temperature operation for the HVAC system and further including an air temperature sensor providing the air temperature within the operational zone;
wherein the dual input thermostat is configured to receive temperature inputs from at least the remote toe temperature sensor and the air temperature sensor and is configured to adjust the set point temperature of the HVAC system relative to the air temperature inversely and proportionately based on the toe temperature provided by the remote toe temperature sensor, the set point temperature being bounded by the range of temperature operation for the HVAC system delimited by the high limit room temperature setting and low limit room temperature setting of the dual input thermostat.

2. The medical device of claim 1, wherein the remote toe temperature sensor in communication with the dual input thermostat is in wireless communication with the dual input thermostat.

3. The medical device of claim 1, wherein the at least one control for the adjustment of the high limit room temperature setting and the low limit room temperature includes a pair of controls including one control for controlling the high limit room temperature setting and one control for controlling the low limit room temperature setting.

4. The medical device of claim 1, wherein the at least one controls are disposed on a housing of the dual input thermostat.

5. The medical device of claim 1, wherein the air temperature sensor is located within the dual input thermostat.

6. The medical device of claim 1, wherein the dual input thermostat is configured such that, when the dual input thermostat receives a temperature input from the toe temperature sensor, an adjustment the set point temperature of the HVAC system relative to the air temperature is time delayed to dampen the response to the toe temperature sensor.

7. The medical device of claim 6, wherein the dual input thermostat further comprises a visual indicator configured to indicate a warning when dampened toe temperature falls below a predetermined value corresponding to the skin activation temperature of locally-mediated vasoconstriction.

8. The medical device of claim 7, wherein the visual indicator is a colored light.

9. The medical device of claim 1, wherein the medical device protects the individual against indoor cold-induced disease, regardless of age of the individual.

10. The medical device of claim 1, wherein the medical device protects the individual against indoor cold-induced hypertension, regardless of age of the individual.

11. The medical device of claim 1, wherein the medical device maintains the indoor heat balance of a body of the individual, with or without the use of behavioral thermoregulation.

12. The medical device of claim 1, wherein the medical device protects the individual against an age-related progression of vasoconstriction, regardless of age of the individual.

13. The medical device of claim 1, wherein the medical device protects the individual against indoor positive feedback vasoconstriction.

14. The medical device of claim 1, wherein the medical device protects the individual against indoor hypothermia.

15. The medical device of claim 1, wherein the medical device connects a closed loop control system of the dual input thermostat with a thermoregulatory closed loop control system of a body of the individual to create a third interactive loop therebetween, while still allowing the closed loop control system and the thermoregulatory closed loop control system to control autonomously.

16. A method of operating the medical device of claim 1, the method comprising the steps of:
measuring the toe temperature using the remote toe temperature sensor and the air temperature using the air temperature sensor;
providing the toe temperature from the remote toe temperature sensor and the air temperature using the air temperature sensor to the dual input thermostat;
adjusting the set point temperature of the HVAC system relative to the air temperature inversely and proportionately based on the toe temperature provided by the remote toe temperature sensor, the set point temperature being bounded by the range of temperature operation for the HVAC system delimited by the high limit room temperature setting and low limit room temperature setting of the dual input thermostat.

17. The method of claim 16, wherein the step of providing the toe temperature from the remote toe temperature sensor to the dual input thermostat involves wirelessly transmitting the toe temperature to the dual input thermostat.

18. The method of claim 16, further comprising the steps of receiving an input from the at least one control to adjust at least one of the high limit room temperature setting and low limit room temperature setting and of adjusting the range of temperature operation for the HVAC system.

19. The method of claim 16, wherein the step of adjusting the set point temperature of the HVAC system relative to the air temperature occurs in response to a change in the toe temperature.

20. The method of claim 19, wherein the step of adjusting is time delayed to dampen the response to the toe temperature sensor.
